(19) United States
(12) Patent Application Publication
(10) Pub. No.: US 2023/0081388 A1
(43) Pub. Date: Mar. 16, 2023

(54) ANTISENSE OLIGOMERS AND METHODS FOR TREATING PARKIN-RELATED PATHOLOGIES

(71) Applicant: MURDOCH UNIVERSITY, Murdoch (AU)

(72) Inventors: Stephen Wilton, Murdoch (AU); May Aung-Htut, Murdoch (AU); Dunhui Li, Como (AU)

(21) Appl. No.: 17/795,828

(22) PCT Filed: Jan. 28, 2021
(86) PCT No.: PCT/AU2021/050054

§ 371 Date: Jul. 27, 2022

(30) Foreign Application Priority Data
Jan. 28, 2020 (AU) 2020900220

(51) Int. Cl. C12N 15/113; A61P 25/16

(52) U.S. Cl. CPC C12N 15/1137; A61P 25/16; C12N 2320/33; C12N 2310/313; C12N 2310/3233; C12N 2310/11; C12N 2310/346; C12N 2310/3513; C12N 2310/321

(57) ABSTRACT

An isolated or purified antisense oligomer with a modified backbone structure for modifying pre-mRNA splicing in the parkin gene transcript or part thereof.

### TECHNICAL FIELD

The present invention relates to the use of antisense oligomers to treat, prevent or ameliorate the effects of common PARK2 mutations causing juvenile Parkinson's disease.

### BACKGROUND ART

Parkinson's disease (PD) is one of the most common neurodegenerative diseases, affecting approximately 1% of the population over the age of 60 years. The main neuropathological findings of PD are the loss of dopaminergic neurons in the substantia nigra pars compacta (SNpc) and the presence of Lewy body and Lewy neurites (α-synuclein polymers) in the SNpc or in other regions of the brain, such as the dorsal motor nucleus of the vagus and locus coeruleus. These pathological distributions result in a wide spectrum of clinical manifestations including core motor and non-motor symptoms. Currently available treatments merely ameliorate PD symptoms. Although some compounds from pre-clinical studies or clinical trials of PD are showing “neuroprotection”, no single intervention exists to slow PD progression or modify the disease course. Hence, a “one-for-all” therapeutic strategy is unlikely to be viable for all PD cases, since many causative genes and molecular pathways are involved in the pathophysiology of this complicated disease.

One of the main pathological hallmarks of PD is the gradual loss of dopaminergic neurons in the SNpc that causes dopamine insufficiency. Dopamine replacement therapy is the primary symptomatic treatment for PD and has existed for nearly 50 years. New drug development pathways for PD include dopamine agonists, monoamine oxidase B inhibitors, or acetylcholinesterase inhibitors. These treatments focus on increasing the activity of dopamine or decreasing the action of its antagonist and have been beneficial for most PD cases. However, the effectiveness of these drugs decreases over time and they only address one aspect of PD pathogenesis. In addition, adverse side effects including dyskinesia, ‘wearing off’ of levodopa, or impulse control disorders of dopamine agonists remain a problem. PD is a complicated disease, where a wide spectrum of symptoms and genetic-molecular pathogenesis are involved, thus new therapeutic strategies, in particular precision medicines tailored for each PD subtype or individual, are needed. Studies including the Parkinson's Disease Biomarker's Program and Parkinson's Progression Markers Initiative are gathering clinical, genetic and biological data to understand disease diversity. Such initiatives will be essential to characterize PD patients according to specific genetic-molecular pathogenesis and identify those potentially amenable to personalized drug development.

Parkin-type autosomal recessive juvenile PD (ARJP) is a form of PD caused by PRKN mutations that accounts for approximately 50% of autosomal recessive Parkinsonism and 15% of early-onset sporadic PD with disease onset often before the age of 45. This PD subtype is clinically characterised by early symmetrical onset, dystonia at onset and hyperreflexia. Patients with Parkin-type ARJP generally show a good response to levodopa; however, motor fluctuations and levodopa-induced dyskinesia are frequently seen during the disease course. Parkin is involved in a wide range of cellular processes and signalling pathways, including mitochondrial homeostasis and apoptosis pathways through ubiquitination of a wide range of substrates. The co-expression of ubiquitin, a-synuclein and a-synuclein interacting protein, synphilin-1, indicates the molecular roles of parkin in the formation of Lewy bodies that might also explain the absence of Lewy body in Parkin-type ARJP cases where parkin is depleted. Parkin is required to ubiquitinate the mitochondrial outer membrane proteins and promote mitophagy to support a healthy mitochondrial network that manages or regulates aging and neurodegeneration through its regulation of cell survival and death. Non-functional parkin leads to mitochondrial swelling, cytochrome c release, and caspase activation, and may indicate the central role of mitochondrial dysfunction in the pathogenesis of Parkin-type ARJP.

Since Lewy body and Lewy neurites are rarely seen in Parkin-type ARJP, this PD subtype is believed to be a distinct clinical entity and shows different pathogenesis compared to the α-synuclein pathology. The PRKN encoded protein, parkin, works as a Ring-Between-Ring (RBR) E3 ligase in the ubiquitin proteasome system and participates in the mitochondrial quality control network to maintain mitochondrial homeostasis. Insufficiency of functional parkin compromises its transcriptional repression of p53 and mediates programmed neuronal cell death and neurodegeneration. Importantly, genotype-phenotype studies on Parkin-type ARJP showed that patients with genomic deletion of both exons 3 and 4 in PRKN present with milder symptoms than patients with the out-of-frame exon 4 deletion.

There is a need to provide an improved or alternative method for treating, preventing or ameliorating the effects of ARJP; or at least the provision of alternative methods to compliment the previously known treatment methods.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides antisense oligonucleotides to induce an internally truncated but functional parkin protein for the treatment of autosomal recessive juvenile onset Parkinson's patients with mutations in the parkin gene.

The present invention provides an isolated or purified antisense oligomer with a modified backbone structure for modifying pre-mRNA splicing in the parkin gene transcript or part thereof.

Preferably the antisense oligomer induces skipping of target exons in the parkin gene transcript or part thereof. Preferably the antisense oligomer is selected from the list comprising: SEQ ID NOs: 1-31.

The present invention further provides a method for manipulating splicing in a parkin gene transcript, the method including the step of:
providing an isolated or purified antisense oligomer with a modified backbone structure for modifying pre-mRNA splicing in the parkin gene transcript or part thereof and combinations or cocktails thereof and allowing the oligomer(s) to bind to a target nucleic acid site.

The present invention further provides a pharmaceutical, prophylactic, or therapeutic composition to treat, prevent or ameliorate the effects of a disease related to parkin expression in a patient, the composition comprising:
an antisense oligomer with a modified backbone structure for modifying pre-mRNA splicing in the parkin gene transcript or part thereof and combinations or cocktails thereof, and
one or more pharmaceutically acceptable carriers and/or diluents.

A method to treat, prevent or ameliorate the effects of a disease associated with parkin expression, comprising the step of:
administering to the patient an effective amount of an antisense oligomer with a modified backbone structure for modifying pre-mRNA splicing in the parkin gene transcript or part thereof and combinations or cocktails thereof, or a pharmaceutical composition comprising the same.

The use of an isolated or purified antisense oligomer with a modified backbone structure for modifying pre-mRNA splicing in the parkin gene transcript or part thereof and combinations or cocktails thereof, for the manufacture of a medicament to treat, prevent or ameliorate the effects of a disease associated with parkin expression.

A kit to treat, prevent or ameliorate the effects of a disease associated with parkin expression in a patient, which kit comprises at least an antisense oligomer with a modified backbone structure for modifying pre-mRNA splicing in the parkin gene transcript or part thereof and combinations or cocktails thereof, packaged in a suitable container, together with instructions for its use.

Preferably the parkin expression related disease is Parkin-type autosomal recessive juvenile Parkinson's disease.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1. Predicted exon splicing enhancers and AO binding sites for PRKN exon 4. Exon splicing enhancers and silencers were predicted by SpliceAid (http://www.introni.it/splicing.html). AO sequences (AO 1-4) were designed to skip PRKN exon 4 by targeting the exon splicing enhancers or exon-intron boundaries. A second generation of AO sequences (AO 5-7) were designed to “Micro-walk” and refine the AO 2 target region identified during evaluation of AOs 1-4.

FIG. 2. PRKN transcript analysis following 2′-O-methyl AO transfection. RNA prepared from fibroblasts from a Parkinson's patient with genomic deletion of exon 3, following transfection with PRKN exon 4 targeting 2′-O-methyl AOs was analysed; (A) RT-PCR analysis of PRKN transcripts from transfected fibroblasts at AO concentration of 100 nM, 50 nM and 25 nM. A scrambled sequence AO was used as a negative control. The 100 bp DNA ladder was used to indicate transcript size, and an RT-PCR no-template negative control was loaded in the last lane; (B) The sequencing chromatogram shows skipping of exon 4 from the transcript encoded by the allele carrying the missense mutation and the absence of exons 3+4 from that with PRKN exon 3 genomic deletion; (C) RT-PCR analysis of transcripts after transfection with “micro-walking” AOs, the original AO sequence amplicons was used to compare the exon skipping efficiency. The SMN transcript was used as a house-keeping reference; (D) Densitometry analysis of the PRKN exons 3+4-skipped product demonstrated a dose response to all the AOs and indicated an AO sequence that induced the highest level of exon skipping.

FIG. 3. Parkin protein structure, PRKN exon map and the induction of the shorter Parkin protein. (A) The normal parkin protein is a RING-between-RING E3 ligase that mainly consists of the Ubiquitin-like domain, RING 0, RING 1, in-between-ring motif (IBR), repressor element of parkin (REP) and RING 2 domain; (B) The parkin gene spans around 1.6 megabases of DNA with 12 average sized exons but huge introns; (C) RT-PCR analysis confirmed the PRKN exon 4 skipping after the PMO treatment in patient fibroblasts at a concentration of 20 μM, Gene Tools control (GTC) sequence was used as a negative control; (D) Western blot shows the induction of the shorter parkin protein induced by PMO (20 μM) treatment in patient fibroblasts.

FIG. 4. Immunofluorescence staining and the relocation of parkin on depolarized mitochondria. Immunofluorescence staining of the parkin protein and Tomm20 to indicate the functionality of the internally truncated parkin protein. Parkin is located uniformly in the cytoplasm in normal human fibroblasts when the mitochondria is healthy. When mitochondria are depolarised by 50 μM carbonyl cyanide m-chlorophenyl hydrazone (CCCP) in healthy human fibroblasts, cytoplasmic normal parkin protein colocalises with the depolarised mitochondria. A similar pattern was observed in the patient fibroblasts after the PMO treatment, while in the Gene Tools control (GTC) PMO treated and no PMO treated patient fibroblasts, the defective parkin was not able to colocalise with the ‘unhealthy’ mitochondria.

FIG. 5. Real-time PCR analysis of p53 expression. The p53 expression level was quantitated by using a relative standard curve and normalized to that in Gene Tools control (GTC) PMO treated patient fibroblasts. Average normalized p53 mRNA expression level for each sample was calculated and graphed. Compared to the GTC PMO treated sample, the p53 mRNA level was decreased around 40% in cells treated with 20 μM of the exon 4 skipping PMO for 72 hours (p=0.02) and around 30% decrease was observed after 10 μM treatment (p=0.03).

FIG. 6. RT-PCR analysis of PRKN transcript following application of AO cocktails to skip exon 3 in healthy human fibroblasts. Singe round RT-PCR was performed to detect PRKN exon 3 skipping. A 100 bp DNA ladder was used to indicate the sizes of the PCR products, an RT-PCR no-template negative control was loaded in the last lane. FL: full length transcript; SC: scramble sequence control; UT: untreated.

FIG. 7. Sanger sequencing results from the patient fibroblasts. The PRKN mutations in a PD patient were confirmed by Sanger sequencing, with an exon 3 deletion detected on one allele and a single base substitution (c.719 C>T) in exon 6 on the second allele. Sanger sequencing used a forward primer in exon 3 that only showed the mutant peak in exon 6, and a reverse primer in exon 8 that revealed the exon 3 deletion.

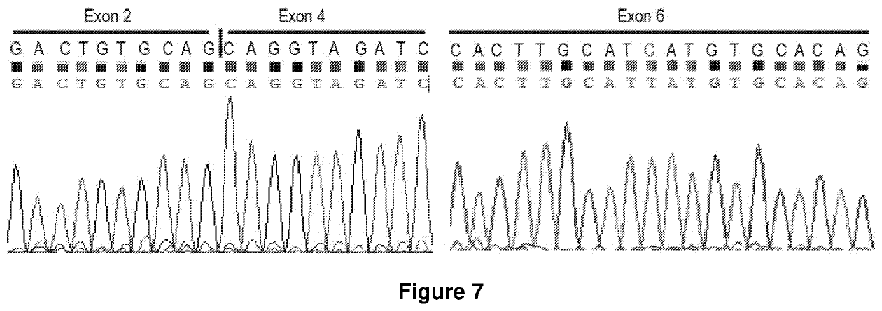

FIG. 8. Immunofluorescence labelling of the parkin protein (green) and Tomm20 (red). Wild-type parkin protein colocalises with the depolarised mitochondria in healthy human fibroblasts after the treatment of 50 μM carbonyl cyanide 3-chlorophenylhydrazone (CCCP) for 3 hours. A similar co-localisation pattern was observed in the PMO-treated patient fibroblasts. However, the defective parkin did not colocalise with the impaired mitochondria in the Gene Tools control (GTC) PMO-treated and untreated patient fibroblasts. Arrows indicate the location of truncated functional parkin protein to the depolarised mitochondria. The fluorescent intensity profiles are shown on the right for each treatment.

### DESCRIPTION OF INVENTION

### Detailed Description of the Invention

### Antisense Oligonucleotides

Parkin-type autosomal recessive juvenile Parkinson's disease is caused by mutations in PRKN, accounting for 50% of all autosomal recessive Parkinsonism cases. Genomic deletions of PRKN exon 3 disrupt the reading frame of the mRNA and result in the loss of functional parkin protein. Parkin is a neuroprotective protein that has dual functions as an E3 ligase in the ubiquitin proteasome system and as a transcriptional repressor of p53. A PRKN genomic deletion of both exons 3 and 4 maintains the reading frame and is associated with a later onset, milder disease progression, indicating this particular isoform retains some function.

Human PRKN is one of the largest human genes. A large number of genetic variations including missense, nonsense, splice site mutations or genomic deletions have been reported across the entire PRKN gene. Furthermore, as a huge gene spanning 1.6 megabases of DNA with only 12 exons, PRKN is located on one of the known fragile sites, FRA6E (6q26) that exhibits genomic instability and is believed to cause several characterized PRKN familial deletions. Clinical phenotype-genotype studies showed that PD patients carrying deletion of PRKN exons 3 and 4 on one allele have milder symptoms than patients with a complete parkin deficiency arising from two null/inactive alleles.

The six domains of parkin form an autoinhibitory conformation whereby the catalytic RING2 is blocked by RING0. Upon phosphorylation by PTEN-induced kinase 1 (PINK1), parkin undergoes a conformational change to expose the RING2 domain and exhibits E3 ligase activity. Several parkin constructs, lacking the Ubl domain; the first 95 aa; or the Ubl plus the following linker domain did not show compromised catalytic activity when compared to normal parkin. The internally truncated parkin protein missing 25% of the Ubl domain, the entire linker domain and 44% of the RING0 domain encoded by exon 3 and 4 was seen to colocalise with depolarised mitochondria in patient cells. Parkin also has a role as a transcriptional repressor of p53, unrelated to its E3 ligase activity, for which the parkin RING1 domain or the RING1 plus the following in-between-ring motif (IBR) domains are essential.

Since a 2-fold difference in p53 mRNA expression level has been detected between cells from the healthy population and those from Parkin-type ARJP, reducing p53 expression by 40% in patients may be expected to bring the p53 level closer to that found in healthy individuals.

The present invention provides antisense oligomers to restore functional parkin expression in cells from Parkinson's patients carrying a heterozygous PRKN exon 3 deletion. For example, antisense oligomer-induced exon 4 skipping may be used to correct the reading frame in the patient cells carrying a frame-shifting exon 3 deletion. The exon skipped, internally truncated re-framed parkin transcript is translated into a shorter but semi-functional parkin isoform that can be recruited to depolarised mitochondria and repress transcription of p53.

The present invention therefore provides antisense oligonucleotides to induce an internally truncated but functional parkin protein.

In contrast to other antisense oligomer-based therapies, the present invention does not induce increased degradation of RNA via recruitment of RNase H. Nor does it rely on hybridization of the antisense oligomer to the parkin genomic DNA or the binding of antisense oligomers to mRNA to modulate the amount of parkin protein produced by interfering with normal functions such as replication, transcription, translocation and translation.

Rather, the antisense oligomers are used to modify pre-mRNA splicing in the primary parkin gene transcript (pre-mRNA) or part thereof and induce exon “skipping” of the target exon/s. The strategy preferably generates an internally truncated but ‘in frame’ mRNA capable of being translated into a protein that retains function.

According to a first aspect of the invention, there is provided antisense oligomer sequences capable of binding to a selected target on a parkin gene transcript to specifically redirect pre-mRNA splicing in a primary parkin gene transcript. Broadly, there is provided an isolated or purified antisense oligomer for inducing targeted exon exclusion in a parkin gene transcript.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An antisense oligomer can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing. The target sequence may be within an exon or within flanking introns or spanning an intron/exon or exon/intron junction.

In certain embodiments, the antisense oligomer has sufficient sequence complementarity to a target RNA (i.e., the RNA for which splice site selection is modulated) to block a region of a target RNA (e.g., pre-mRNA) in an effective manner. In exemplary embodiments, such blocking of parkin pre-mRNA serves to modulate splicing, either by masking a binding site for a native protein that would otherwise modulate splicing and/or by altering the structure of the targeted RNA. In some embodiments, the target RNA is target pre-mRNA (e.g., parkin gene pre-mRNA).

An antisense oligomer having a sufficient sequence complementarity to a target RNA sequence to modulate splicing of the target RNA means that the antisense oligomer has a sequence sufficient to trigger the masking of a binding site for a native protein or nucleic acid that would otherwise modulate splicing and/or alters the three-dimensional structure of the targeted RNA.

Selected antisense oligomers can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a melting temperature (Tm) of 45° C. or greater.

Preferably, the antisense oligomer is selected from the group comprising the sequences set forth in Table 1. Preferably, the antisense oligomer is selected from the group comprising the sequences in SEQ ID NOs: 1-31, more preferably SEQ ID NOs: 11-14, 24-26 and 31, even more preferably SEQ ID NOs: 24-26 and 31.

In certain embodiments, the degree of complementarity between the target sequence and antisense oligomer is sufficient to form a stable duplex. The region of complementarity of the antisense oligomers with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An antisense oligomer of about 16-17 bases is generally long enough to have a unique complementary sequence. In certain embodiments, a minimum length of complementary bases may be required to achieve the requisite binding Tm, as discussed herein.

In certain embodiments, oligomers as long as 50 bases may be suitable, where at least a minimum number of bases, e.g., 10-12 bases, are complementary to the target sequence. In general, however, facilitated or active uptake in cells is optimized at oligomer lengths of less than about 30 bases. For phosphorodiamidate morpholino oligomer (PMO) antisense oligomers, an optimum balance of binding stability and uptake generally occurs at lengths of 18-35 bases. Included are antisense oligomers (e.g., CPP-PMOs, PPMOs, PMOs, PMO-X, PMO+ PNAs, LNAs, 2′-OMe, 2′MOE) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation that may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing of the target pre-RNA can be modulated.

The stability of the duplex formed between an antisense oligomer and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included.

Additional examples of variants include antisense oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of in SEQ ID NOs: 1-31, more preferably SEQ ID NOs: 11-14, 24-26 and 31, even more preferably SEQ ID NOs: 24-26 and 31 or the sequences provided in Table 1.

More specifically, there is provided an antisense oligomer capable of binding to a selected target site to modify pre-mRNA splicing in a parkin gene transcript or part thereof. The antisense oligomer is preferably selected from those provided in Table 1 or in SEQ ID NOs: 1-31, more preferably SEQ ID NOs: 11-14, 24-26 and 31, even more preferably SEQ ID NOs: 24-26 and 31.

The modification of pre-mRNA splicing preferably induces “skipping”, or the removal of one or more exons or introns of the mRNA. The resultant protein may be of a shorter length when compared to the parent full-length parkin protein due to either internal truncation or premature termination. These parkin proteins may be termed isoforms of the unmodified parkin protein.

The remaining exons of the mRNA generated may be in-frame and produce a shorter protein with a sequence that is similar to that of the parent full length protein, except that it has an internal truncation in a region between the original 3′ and 5′ ends.

Functional domains of parkin include the ubiquitin-like domain, RING1, in-between-region, and the RING2 domain. Exclusion of exons 3+4 removes a small part of the ubiquitin-like domain, the linker and a part of the RING0 domain, but will not disrupt the open reading frame, leading to a shortened transcript and internally truncated protein that retains most of the parkin functions.

The skipping process of the present invention, using antisense oligomers, may skip an individual exon, or may result in skipping two or more exons at once.

The antisense oligomers of the invention may be a combination of two or more antisense oligomers capable of binding to a selected target to induce exon exclusion in a parkin gene transcript. The combination may be a cocktail of two or more antisense oligomers and/or a construct comprising two or more or two or more antisense oligomers joined together.

Alternatively, the antisense oligomer may comprise a sequence designed to target two or more discontinuous regions in one exon. For example, such sequences may comprise a first antisense oligomer sequence and a second antisense oligomer sequence directed to a nearby splice motif, in the same antisense oligomer transcript. Such antisense oligomers may be named such that they have two or three locations in their name (see, for example SEQ ID NOs: 27-30). Preferably such antisense oligomers with sequence directed to target two or more discontinuous regions in one exon are locked nucleic acids (LNAs).

TABLE 1
List of antisense oligonucleotide sequences used in this study

| Seq | | | Length |
|---|---|---|---|
| ID | AO nomenclature | Sequence 5′→3′ | (bp) |
| 1 | PRKN_H2A(−16+7) | ACAAACACUGACCAAGGAAAUUG | 23 |
| 2 | PRKN_H2A(+51+78) | CUCCUUGAGCUGGAAGAUGCUGGUGUC | 27 |
| 3 | PRKN_H2A(+116+141) | CUCCUUCCCUGCGAAAAUCACACGCA | 26 |
| 4 | PRKN_H2D(+22−4) | UCACCUGCACAGUCCAGUCAUUCCUC | 26 |
| 5 | PRKN_H2A(+10+33) | GGAAACCAUGGCUGGAGUUGAACC | 24 |
| 6 | PRKN_H3A(+9+35) | AUGUGAACAAUGCUCUGCUGAUCCAGG | 27 |
| 7 | PRKN_H3A(+205+230) | CUGGUGGUGAGUCCUUCCUGCUGUCA | 25 |
| 8 | PRKN_H3A(−5+22) | UCUGCUGAUCCAGGUCACAAUUCUGUU | 27 |
| 9 | PRKN_H3D(−5+21) | AUUACCUGGACUUCCAGCUGGUGGUG | 25 |
| 10 | PRKN_H3A(+137+161) | CUGAGCUGCUGAGGUCCACCCGAGU | 25 |
| 11 | PRKN_H4A(−15+10) | GAUCUACCUGCUGGAGAAGAAAAAG | 25 |
| 12 | PRKN_H4A(+7+33) | ACACAUAAAAGCUGUUGUAGAUUGAUC | 27 |
| 13 | PRKN_H4A(+86+114) | GGUGAGCGUUGCCUGCCUGCAGGUGCUGC | 29 |
| 14 | PRKN_H4D(−17+8) | ACACUGCAUUUCCUUACCUGGGUCA | 25 |
| 15 | PRKN_H2A(+111+136) | UCCCUGCGAAAAUCACACGCAACUGG | 26 |
| 16 | PRKN_H2A(+121+146) | CUCAGCUCCUUCCCUGCGAAAAUCAC | 26 |
| 17 | PRKN_H2A(+118+139) | CCUUCCCUGCGAAAAUCACACG | 22 |
| 18 | PRKN_H3A(+36+60) | ACCUUUUCUCCACGGUCUCUGCACA | 25 |
| 19 | PRKN_H3A(+62+86) | UCGCCUCCAGUUGCAUUCAUUUCUU | 25 |
| 20 | PRKN_H3A(+50+69) | CAUUUCUUGACCUUUUCUCC | 20 |
| 21 | PRKN_H3A(+55+81) | CCAGUUGCAUUCAUUUCUUGACCUUU | 26 |
| 22 | PRKN_H3A(+195+219) | GUCCUUCCUGCUGUCAGUGUGCAGA | 25 |
| 23 | PRKN_H3D(−20+5) | UCUUAGAGCAUUCCAAUUACCUGGA | 25 |
| 24 | PRKN_H4A(+2+28) | UAAAAGCUGUUGUAGAUUGAUCUACCU | 27 |
| 25 | PRKN_H4A(+12+38) | GCAAUACACAUAAAAGCUGUUGUAGAU | 27 |
| 26 | PRKN_H4A(+13+34) | UACACAUAAAAGCUGUUGUAGA | 22 |
| 27 | PRKN_H3A | CUGAUCCAGCGCCUCCAGAGUGUGCAG | 27 |
| | (+26+34, +63+71, +209+218) | | |
| 28 | PRKN_H3A(+20+32, +63+75) | GCUCUGCUGAUCCCGCCUCCAGUUG | 25 |
| 29 | PRKN_H3A(+63+75, +201+213) | CGCCUCCAGUUGCCUGCUGUCAGUG | 25 |
| 30 | PRKN_H3A(+20+31, +201+213) | GCUCUGCUGAUCCCUGCUGUCAGUG | 25 |
| 31 | PRKN_H4A(+13+34) | TACACATAAAAGCTGTTGTAGA | 22 |

There is also provided a method for manipulating splicing in a parkin gene transcript, the method including the step of:
a) providing one or more of the antisense oligomers as described herein and allowing the oligomer(s) to bind to a target nucleic acid site.

According to yet another aspect of the invention, there is provided a splice manipulation target nucleic acid sequence for parkin comprising the DNA equivalents of the nucleic acid sequences selected from Table 1 or the group consisting of in SEQ ID NOs: 1-31, more preferably SEQ ID NOs: 11-14, 24-26 and 31, even more preferably SEQ ID NOs: 24-26 and 31, and sequences complementary thereto.

Designing antisense oligomers to completely mask consensus splice sites may not necessarily generate a change in splicing of the targeted exon. Furthermore, the inventors have discovered that size or length of the antisense oligomer itself is not always a primary factor when designing antisense oligomers. With some targets such as ITGA4 exon 3, antisense oligomers as short as 20 bases were able to induce some exon skipping, in certain cases more efficiently than other longer (eg 25 bases) oligomers directed to the same exon.

The inventors have also discovered that there does not appear to be any standard motif that can be blocked or masked by antisense oligomers to redirect splicing. It has been found that antisense oligomers must be designed and their individual efficacy evaluated empirically.

More specifically, the antisense oligomer may be selected from those set forth in Table 1. The sequences are preferably selected from the group consisting of any one or more of any one or in SEQ ID NOs: 1-31, more preferably SEQ ID NOs: 11-14, 24-26 and 31, even more preferably SEQ ID NOs: 24-26 and 31, and combinations or cocktails thereof. This includes sequences that can hybridise to such sequences under stringent hybridisation conditions, sequences complementary thereto, sequences containing modified bases, modified backbones, and functional truncations or extensions thereof which possess or modulate pre-mRNA processing activity in a parkin gene transcript.

The oligomer and the DNA, cDNA or RNA are complementary to each other when a sufficient number of corresponding positions in each molecule are occupied by nucleotides that can hydrogen bond with each other. Thus, “specifically hybridisable” and “complementary” are terms that are used to indicate a sufficient degree of complementarity or pairing such that stable and specific binding occurs between the oligomer and the DNA, cDNA or RNA target. It is understood in the art that the sequence of an antisense oligomer need not be 100% complementary to that of its target sequence to be specifically hybridisable. An antisense oligomer is specifically hybridisable when binding of the compound to the target DNA or RNA molecule interferes with the normal function of the target DNA or RNA product, and there is a sufficient degree of complementarity to avoid non-specific binding of the antisense oligomer to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the antisense oligomers of the present invention may include oligomers that selectively hybridise to the sequences provided in Table 1, or in SEQ ID NOs: 1-31, more preferably SEQ ID NOs: 11-14, 24-26 and 31, even more preferably SEQ ID NOs: 24-26 and 31.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligomers may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the antisense oligomer sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an antisense oligomer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligomers set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The antisense oligomers of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or antisense oligomers or compositions to produce or cause a greater physiological response (i.e., downstream effects) in a cell or a subject relative to the response caused by either no antisense oligomer or a control compound. The terms “decreasing” or “decrease” refer generally to the ability of one or more antisense oligomers or compositions to produce or cause a reduced physiological response (i.e., downstream effects) in a cell or a subject relative to the response caused by either no antisense oligomer or a control compound.

Relevant physiological or cellular responses (in vivo or in vitro) will be apparent to persons skilled in the art, and may include increases in the exclusion of specific exons in a parkin-coding pre-mRNA, increase in the amount of parkin-coding pre-mRNA or increases in the expression of functional parkin protein in a cell, tissue, or subject in need thereof. An “decreased” or “reduced”, or increased amount is typically a statistically significant amount, and may include a decrease or increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) more or less than the amount produced when no antisense oligomer is present (the absence of an agent) or a control compound is used.

The term “reduce” or “inhibit” may relate generally to the ability of one or more antisense oligomers or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as Parkin-type autosomal recessive juvenile Parkinson's disease.

A “decrease” in a response may be statistically significant as compared to the response produced by no antisense oligomer or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligomer may vary, as long as it is capable of binding selectively to the intended location within the pre-mRNA molecule. The length of such sequences can be determined in accordance with selection procedures described herein. Generally, the antisense oligomer will be from about 10 nucleotides in length, up to about 50 nucleotides in length. It will be appreciated, however, that any length of nucleotides within this range may be used in the method. Preferably, the length of the antisense oligomer is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “antisense oligomer” refers to a linear sequence of nucleotides, or nucleotide analogs, that allows the nucleobase to hybridize to a target sequence in an RNA by Watson-Crick base pairing, to form an oligomer: RNA heteroduplex within the target sequence. The terms “antisense oligomer”, “antisense oligonucleotide”, “oligomer” and “antisense compound” may be used interchangeably to refer to an oligonucleotide or oligonucleotide analogue. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), 2′-O-methyl and 2′-O-methoxyethyl oligonucleotides, among other antisense agents known in the art.

Included are non-naturally-occurring antisense oligomers, or “oligonucleotide analogs”, including antisense oligomers or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing antisense oligomers is the alkylation of the 2′ hydroxyribose position and the incorporation of a phosphorothioate backbone to produce molecules that superficially resemble RNA but that are much more resistant to nuclease degradation, although persons skilled in the art of the invention will be aware of other forms of suitable backbones that may be useable in the objectives of the invention.

To avoid degradation of pre-mRNA during duplex formation with the antisense oligomers, the antisense oligomers used in the method may be adapted to minimise or prevent cleavage of the target RNA by endogenous RNase H. This property is highly preferred, as the treatment of the RNA with the unmethylated oligomers, either intracellular or in crude extracts that contain RNase H, leads to degradation of the RNA strand of the pre-mRNA: antisense oligomer duplexes. Any form of modified antisense oligomers that is capable of by-passing or not inducing such degradation may be used in the present method. The nuclease resistance may be achieved by modifying the antisense oligomers of the invention so that it comprises partially unsaturated aliphatic hydrocarbon chain and one or more polar or charged groups including carboxylic acid groups, ester groups, and alcohol groups.

Antisense oligomers that do not activate RNase H can be made in accordance with known techniques (see, e.g., U.S. Pat. No. 5,149,797). Such antisense oligomers that may be deoxyribonucleotide or ribonucleotide sequences, simply contain any structural modification that sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous antisense oligomers that do not activate RNase H are available. For example, such antisense oligomers may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such antisense oligomers are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

An example of antisense oligomers that when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant antisense oligomers of the invention may have at least one of the last 3′-terminus nucleotides fluoridated. Still alternatively, the nuclease resistant antisense oligomers of the invention have phosphorothioate bonds linking between at least two of the last 3-terminus nucleotide bases, preferably having phosphorothioate bonds linking between the last four 3′-terminal nucleotide bases.

Increased splice-switching may also be achieved with alternative oligonucleotide chemistry. For example, the antisense oligomer may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-methyl-modified oligomer (2′-OMe); 2′-O-methoxy ethyl (2′-MOE); 2′-fluoro, 2′-fluroarabino (FANA); unlocked nucleic acid (UNA); hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping antisense oligomers. Antisense oligomer-induced splice modification of the human parkin gene transcripts has generally used either oligoribonucleotides, PNAs, 2′OMe or MOE modified bases on a phosphorothioate backbone. Although 2′OMeAOs are used for oligo design, due to their efficient uptake in vitro when delivered as cationic lipoplexes, these compounds are susceptible to nuclease degradation and are not considered ideal for systemic in vivo or clinical applications. When alternative chemistries are used to generate the antisense oligomers of the present invention, the uracil (U) of the sequences provided herein may be replaced by a thymine (T).

While the antisense oligomers described above are a preferred form of the antisense oligomers of the present invention, the present invention includes other oligomeric antisense molecules, including but not limited to oligomer mimetics such as are described below.

Specific examples of preferred antisense oligomers useful in this invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be antisense oligomers.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds that are not degraded by any known nuclease or protease. These compounds are uncharged, do not activate RNase H activity when annealed to an RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. Incorporation of 5-methylcytosine bases has been shown to increase nucleic acid duplex stability by 0.6-1.2° C., even more particularly when combined with 2′-O-methoxyethyl sugar modifications.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligomer. The present invention also includes antisense oligomers that are chimeric compounds. “Chimeric” antisense oligomers or “chimeras,” in the context of this invention, are antisense oligomers, particularly oligomers that contain two or more chemically distinct regions, each made up of at least one monomer unit, i.e., a nucleotide in the case of an oligomer compound. These oligomers typically contain at least one region wherein the oligomer is modified so as to confer upon the oligomer or antisense oligomer increased resistance to nuclease degradation, increased cellular uptake, and an additional region for increased binding affinity for the target nucleic acid.

The activity of antisense oligomers and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide that is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using reverse transcription followed by quantitative PCR to assess the level of expression of the transcript(s).

The present invention provides antisense oligomer induced splice-switching of the parkin gene transcript, using clinically applicable oligomer chemistries and delivery systems to direct parkin splice manipulation to therapeutic levels. Substantial increase in the amount of induced parkin mRNA, and hence parkin protein from parkin gene transcription, are achieved by:
1) oligomer refinement in vitro using fibroblasts, through experimental assessment of (i) exonic and intronic enhancer target motifs, (ii) antisense oligomer length and development of oligomer cocktails, (iii) choice of chemistry, and (iv) the addition of cell-penetrating peptides (CPP) or other moieties to enhance oligomer delivery to cells and tissues; and
2) detailed evaluation of a novel approach to generate parkin transcripts with one or more missing exons.

As such, it is demonstrated herein that processing of parkin pre-mRNA can be manipulated with specific antisense oligomers. In this way functionally significant amounts of parkin protein can be obtained, thereby reducing the severe pathology associated with juvenile onset Parkinson's disease.

The antisense oligomers used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligomers of the invention are synthesised in vitro and include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules etc oral, rectal, topical or other formulations, for assisting in uptake, distribution and/or absorption.

The antisense oligomers may be formulated for oral, topical, parenteral or other delivery, particularly formulations for topical and injectable delivery. The formulations may be formulated for assisting in uptake, distribution and/or absorption at the site of delivery or activity.

Also included are vector delivery systems that are capable of expressing the oligomeric, parkin-targeting sequences of the present invention, such as vectors that express a polynucleotide sequence comprising any one or more of SEQ ID NOs: 11-14, 24-26 and 31, as described herein. By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

### Method of Treatment

The antisense oligomers of the present invention also can be used as a prophylactic or therapeutic, which may be utilised for the purpose of treatment of a disease. Accordingly, in one embodiment the present invention provides antisense oligomers that bind to a selected target in the parkin pre-mRNA to modify splicing of the RNA as described herein, in a therapeutically effective amount, admixed with a pharmaceutically acceptable carrier, diluent, or excipient.

According to a still further aspect of the invention, there is provided one or more antisense oligomers as described herein for use in an antisense oligomer-based therapy. Preferably, the therapy is for a condition related to parkin expression. More preferably, the therapy for a condition related to parkin expression is therapy for Parkin-type autosomal recessive juvenile Parkinson's disease.

More specifically, the antisense oligomer may be selected from Table 1, or the group consisting of any one or more of in SEQ ID NOs: 1-31, more preferably SEQ ID NOs: 11-14, 24-26 and 31, even more preferably SEQ ID NOs: 24-26 and 31, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a parkin gene transcript.

The invention extends also to a combination of two or more antisense oligomers capable of binding to a selected target to induce exon exclusion in a parkin gene transcript. The combination may be a cocktail of two or more antisense oligomers, a construct comprising two or more antisense oligomers joined together for use in an antisense oligomer-based therapy.

There is therefore provided a method to treat, prevent or ameliorate the effects of a disease associated with parkin expression, comprising the step of:
a) administering to the patient an effective amount of one or more antisense oligomers or pharmaceutical composition comprising one or more antisense oligomers as described herein.

Preferably the disease associated with parkin expression in a patient is Parkin-type autosomal recessive juvenile Parkinson's disease.

Therefore, the invention provides a method to treat, prevent or ameliorate the effects of Parkin-type autosomal recessive juvenile Parkinson's disease, comprising the step of:
a) administering to the patient an effective amount of one or more antisense oligomers or pharmaceutical composition comprising one or more antisense oligomers as described herein.

Preferably, the therapy is used to induce a functional parkin protein isoform via an exon skipping strategy tailored to amenable PRKN mutations. The induction of the parkin isoform/s is preferably achieved through modifying pre-mRNA splicing in the parkin gene transcript.

The induction of parkin isoforms will preferably lead to a reduction in the quantity, duration or severity of the symptoms of a parkin-related condition or pathology, such as Parkin-type autosomal recessive juvenile Parkinson's disease.

An “effective amount” or “therapeutically effective amount” refers to an amount of therapeutic compound, such as an antisense oligomer, administered to a mammalian subject, either as a single dose or as part of a series of doses that is effective to produce a desired therapeutic effect.

Treatment may be monitored, e.g., by general indicators of disease known in the art. As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments that can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

A “subject” as used herein, includes any animal that exhibits a symptom, or is at risk for exhibiting a symptom that can be treated with an antisense compound of the invention, or any of the symptoms associated with these conditions. The subject with the disease associated with parkin deficiency may be a mammal, including a human. Other suitable subjects include laboratory animals (such as mouse, rat, rabbit, or guinea pig), farm animals, and domestic animals or pets (such as a cat or dog). Non-human primates and, preferably, human subjects, are included.

The antisense oligomers of the present invention may also be used in conjunction with alternative therapies, such as drug therapies.

The present invention therefore provides a method of treating, preventing or ameliorating the effects of a disease or condition associated with deficient functional parkin expression, wherein the antisense oligomers of the present invention and administered sequentially or concurrently with another alternative therapy associated with treating, preventing or ameliorating the effects of a disease or condition associated with parkin deficiency. Preferably, the disease or condition is Parkin-type autosomal recessive juvenile Parkinson's disease.

### Delivery

The antisense oligomers of the present invention also can be used as a prophylactic or therapeutic that may be utilised for the purpose of treatment of a disease. Accordingly, in one embodiment the present invention provides antisense oligomers that bind to a selected target in the parkin pre-mRNA to induce efficient and consistent exon skipping as described herein, in a therapeutically effective amount, admixed with a pharmaceutically acceptable carrier, diluent, or excipient.

There is also provided a pharmaceutical, prophylactic, or therapeutic composition to treat, prevent or ameliorate the effects of a disease related to deficient parkin expression in a patient, the composition comprising:
a) one or more antisense oligomers as described herein; and
b) one or more pharmaceutically acceptable carriers and/or diluents.

In one embodiment, the antisense oligomer is administered in an amount and manner effective to result in a peak blood concentration of at least 200-400 nM antisense oligomer. Typically, one or more doses of antisense oligomer are administered, initially at regular intervals as a loading dose for a period of about one to two weeks or months and then as required to maintain clinically therapeutic levels. Preferred doses for oral administration are from about 0.01 mg to 200 mg oligomer per kg. For intra venous administration, preferred doses are from about 0.5 mg to 100 mg oligomer per kg. For intra venous or sub cutaneous administration, the antisense oligomer may be administered at a dosage of about up to 200 mg/kg weekly. For intrathecal administration, lower dosage of about 10-30 mg may be administered.

The antisense oligomer may be administered at regular intervals for a short time period, e.g., daily. However, in many cases the oligomer is administered intermittently over a longer period of time. Administration may be followed by, or concurrent with, administration of a small molecule drug eg antibiotic, corticosteroid or other therapeutic treatment. The treatment regimen may be adjusted (dose, frequency, route, etc.) as indicated, based on the results of immunoassays, biomarker assays other biochemical tests and physiological examination of the subject under treatment.

Dosing may be dependent on severity and responsiveness of the disease state to be treated, with the course of treatment lasting from several days to years, or until a cure is effected or a diminution of the disease state is achieved. Alternatively, dosing may be titrated against disease progression rate. A baseline progression is expected to be established on a case-by-case basis. Then the progression rate after an initial once off dose is monitored to check that there is a reduction in the rate. Preferably, there is no progression after dosing. Successful treatment preferably results in no further progression of the disease or even some recovery. Optimal dosing schedules can be calculated from measurements of drug accumulation in the body of the patient. Persons of ordinary skill can easily determine optimum dosages, dosing methodologies and repetition rates.

Optimum dosages may vary depending on the relative potency of individual oligomers and can generally be estimated based on EC50s found to be effective in in vitro and in vivo animal models.

In general, dosage is from 0.01 μg to 200 mg per kg of body weight, and may be given once or more daily, weekly, monthly or yearly, or even less frequently. Persons of ordinary skill in the art can easily estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligomer is administered in maintenance doses, ranging from 0.01 μg to 200 mg per kg of body weight, once or more daily, to once every 20 years.

An effective in vivo treatment regimen using the antisense oligomers of the invention may vary according to the duration, dose, frequency and route of administration, as well as the condition of the subject under treatment (i.e., prophylactic administration versus administration in response to diagnosed ARJP). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligomers of the invention may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

Intranuclear oligomer delivery is a major challenge for antisense oligomers. Different cell-penetrating peptides (CPP) localize PMOs to varying degrees in different conditions and cell lines and tissues, and novel CPPs have been evaluated by the inventors for their ability to deliver PMOs to the target cells. The terms CPP or “a peptide moiety that enhances cellular uptake” are used interchangeably and refer to cell penetrating peptides, also called “transport peptides”, “carrier peptides”, or “peptide transduction domains”. The peptides, as shown herein, have the capability of inducing cell penetration within about or at least about 10%, 20%, 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 2010/0016215, which is incorporated by reference in its entirety.

The present invention therefore provides antisense oligomers of the present invention in combination with cell-penetrating peptides for manufacturing therapeutic pharmaceutical compositions.

### Excipients

In a form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, Pa. 18042) pages 1435-1712 that are herein incorporated by reference. The compositions may be prepared in liquid form, or may be in dried powder, such as a lyophilised form.

The antisense oligomers of the present invention are preferably delivered in a pharmaceutically acceptable composition. The composition may comprise about 1 nM to 1000 nM of each of the desired antisense oligomer(s) of the invention. Preferably, the composition may comprise about 1 nM to 500 nM, 10 nM to 500 nM, 50 nM to 750 nM, 10 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the antisense oligomer(s) of the invention.

The composition may comprise about 1 nm, 2 nm, 3 nm, 4 nm, 5 nm, 6 nm, 7 nm, 8 nm, 9 nm, 10 nm, 20 nm, 50 nm, 75 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm or 1000 nm of each of the desired antisense oligomer(s) of the invention.

The present invention further provides one or more antisense oligomers adapted to aid in the prophylactic or therapeutic treatment, prevention or amelioration of symptoms of a disease such as a parkin expression related disease or pathology in a form suitable for delivery to a patient.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Remington: The Science and Practice of Pharmacy, 22nd Ed., Pharmaceutical Press, PA (2013).

In a more specific form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Remington: The Science and Practice of Pharmacy, 22nd Ed., Pharmaceutical Press, PA (2013). The compositions may be prepared in liquid form, or may be in dried powder, such as a lyophilised form.

It will be appreciated that pharmaceutical compositions provided according to the present invention may be administered by any means known in the art. The pharmaceutical compositions for administration are administered by injection, orally, topically or by the pulmonary or nasal route. For example, the antisense oligomers may be delivered by intravenous, intra-arterial, intraperitoneal, intramuscular or subcutaneous routes of administration. The appropriate route may be determined by one of skill in the art, as appropriate to the condition of the subject under treatment. Vascular or extravascular circulation, the blood or lymph system, and the cerebrospinal fluid are some non-limiting sites where the antisense oligomer may be introduced. Direct CNS delivery may be employed, for instance, intracerebral ventricular or intrathecal administration may be used as routes of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the antisense oligomers of the disclosure can be delivered by topical or transdermal methods (e.g., via incorporation of the antisense oligomers into, e.g., emulsions, with such antisense oligomers optionally packaged into liposomes) including delivery to skin surfaces. Such topical or transdermal and emulsion/liposome-mediated methods of delivery are described for delivery of antisense oligomers in the art, e.g., in U.S. Pat. No. 6,965,025.

The antisense oligomers described herein may also be delivered via an implantable device. Design of such a device is an art-recognized process, with, e.g., synthetic implant design described in, e.g., U.S. Pat. No. 6,969,400. Preferably the implant is able to be implanted into the body for sustained delivery of the antisense oligomers.

Compositions and formulations for oral administration include powders or granules, microparticulates, nanoparticulates, suspensions or solutions in water or non-aqueous media, capsules, gel capsules, sachets, tablets or minitablets. Thickeners, flavouring agents, diluents, emulsifiers, dispersing aids or binders may be desirable. Oral formulations are those in which oligomers of the disclosure are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860. Oral formulations for oligomers and their preparation are described in detail in U.S. Pat. No. 6,887,906, Ser. No. 09/315,298 filed May 20, 1999 and/or US20030027780.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of antisense oligomers may be achieved by methods previously published. For example, delivery of the antisense oligomer may be via a composition comprising an admixture of the antisense oligomer and an effective amount of a block copolymer. An example of this method is described in US patent application US20040248833. Other methods of delivery of antisense oligomers to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

It may be desirable to deliver the antisense oligomer in a colloidal dispersion system. Colloidal dispersion systems include macromolecule complexes, nanocapsules, nanoparticles, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles that are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligomer of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The antisense oligomers described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

As known in the art, antisense oligomers may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomer may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281 ; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The antisense oligomers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be via topical (including ophthalmic and mucous membranes, as well as rectal delivery), pulmonary (e.g., by inhalation or insufflation of powders or aerosols, including by nebulizer, intratracheal, intranasal, epidermal and transdermal), oral or parenteral routes. Parenteral administration includes intravenous, intra-arterial, subcutaneous, intraperitoneal, intraocular or intramuscular injection or infusion; or intracranial, e.g., intrathecal or intraventricular, administration. Oligomers with at least one 2′-O-methoxyethyl modification are believed to be particularly useful for oral administration. Preferably, the antisense oligomer is delivered via the subcutaneous or intravenous route.

The pharmaceutical formulations of the present invention that may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

### Use

According to another aspect of the invention there is provided the use of one or more antisense oligomers as described herein in the manufacture of a medicament for the modulation or control of a disease associated with deficient functional parkin expression.

The invention also provides for the use of purified and isolated antisense oligomers as described herein, for the manufacture of a medicament for treatment of a disease associated with parkin expression.

There is also provided the use of purified and isolated antisense oligomers as described herein, for the manufacture of a medicament to treat, prevent or ameliorate the effects of a disease associated with parkin expression.

Preferably, the parkin-related pathology or disease is Parkin-type autosomal recessive juvenile Parkinson's disease.

The invention extends, according to a still further aspect thereof, to cDNA or cloned copies of the antisense oligomer sequences of the invention, as well as to vectors encoding the antisense oligomer sequences of the invention. The invention extends further also to cells containing such sequences and/or vectors.

### Kits

There is also provided a kit to treat, prevent or ameliorate the effects of a disease associated with parkin expression in a patient, which kit comprises at least an antisense oligomer as described herein and combinations or cocktails thereof, packaged in a suitable container, together with instructions for its use.

In a preferred embodiment, the kits will contain at least one antisense oligomer as described herein or as shown in Table 1, or in SEQ ID NOs: 1-31, more preferably SEQ ID NOs: 11-14, 24-26 and 31, even more preferably SEQ ID NOs: 24-26 and 31, or a cocktail of antisense oligomers, as described herein. The kits may also contain peripheral reagents such as buffers, stabilizers, etc.

There is therefore provided a kit to treat, prevent or ameliorate a disease or condition associated with parkin expression in a patient, which kit comprises at least an antisense oligomer described herein or as shown in Table 1 and combinations or cocktails thereof, packaged in a suitable container, together with instructions for its use.

There is also provided a kit to treat, prevent or ameliorate a disease or condition associated with parkin expression in a patient, which kit comprises at least an antisense oligomer selected from the group consisting of any one or more of in SEQ ID NOs: 1-31, more preferably SEQ ID NOs: 11-14, 24-26 and 31, even more preferably SEQ ID NOs: 24-26 and 31, and combinations or cocktails thereof, packaged in a suitable container, together with instructions for its use.

Preferably, the disease or condition is Parkin-type autosomal recessive juvenile Parkinson's disease.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

In an embodiment, the kit of the present invention comprises a composition comprising a therapeutically effective amount of an antisense oligomer capable of binding to a selected target on a parkin gene transcript to modify pre-mRNA splicing in a parkin gene transcript or part thereof. In an alternative embodiment, the formulation is in pre-measured, pre-mixed and/or pre-packaged. Preferably, the solution is sterile.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the formulations of the present invention. In one embodiment, the instructions indicate that the formulation of the present invention is suitable for the treatment of Parkin-type autosomal recessive juvenile Parkinson's disease. Such instructions may also include instructions on dosage, as well as instructions for administration via injection, orally, topically or by the pulmonary or nasal route.

The antisense oligomers and suitable excipients can be packaged individually so to allow a practitioner or user to formulate the components into a pharmaceutically acceptable composition as needed. Alternatively, the antisense oligomers and suitable excipients can be packaged together, thereby requiring de minimus formulation by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

Sequence identity numbers (“SEQ ID NO:”) containing nucleotide and amino acid sequence information included in this specification are collected at the end of the description and have been prepared using the program PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense oligomer nomenclature system was proposed and published to distinguish between the different antisense oligomers (see Mann et al., (2002) J Gen Med 4, 644-654). This nomenclature became especially relevant when testing several slightly different antisense oligomers, all directed at the same target region, as shown below:
H # A/D (x:y)
the first letter designates the species (e.g. H: human, M: murine)
“#” designates target exon number
“A/D” indicates acceptor or donor splice site at the beginning and end of the exon, respectively
(x y) represents the annealing coordinates where “−” or “+” indicate intronic or exonic sequences respectively. As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense oligomer. Entirely exonic annealing coordinates that would be represented by A(+65+85), that is the site between the 65th and 85th nucleotide, inclusive, from the start of that exon.
The nomenclature was subsequently revised to include the gene name:
HGENE # A/D (x:y)

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Development of AOs

### Methods and Materials

### AO Design and Synthesis

AOs were designed to target PRKN exon 4 acceptor or donor splice sites, and exon splicing enhancers as predicted by the online splice prediction tool: SpliceAid (https://www.introni.it/splicing.html). AOs composed of 2′-O-methyl modified bases on a phosphorothioate backbone were synthesised in-house on an Expedite 8909 Nucleic Acid synthesiser with reagents from Azco Biotech (California, USA). AO nomenclature is according to the descriptions by Mann, et al. 2002, and provides a description of annealing coordinates within the targeted exon, with the addition of the gene name. After initial screening, the most promising AO sequences were optimised by shuffling the annealing sequence several bases upstream or downstream. The sequence of the optimal AO was synthesised as a PMO by Genetools, LLC (Philomath, Oreg., USA).

### Cell Propagation

Parkin-type ARJP patient dermal fibroblasts and normal human primary skin fibroblasts were derived from skin biopsies (Human Ethics Committee of Murdoch University approval 2013/156). Cells were proliferated in Dulbecco's Modified Eagle Medium (DMEM) (Gibco, Life Technologies, Melbourne, Australia) supplemented with 15% fetal bovine serum (FBS) (Serana, Bunbury, Australia) in 75 cm² flasks at 37° C. in a 5% CO₂ atmosphere before seeding into 24-well plates at the cell density of 15,000 per well for RNA extraction or 10,000 per cover slip for staining. Cells were cultured in 10% FBS DMEM for 24 hours before transfection.

### Transfection

Cells were transfected with 2′OMe AOs: Lipofectamine 3000 (L3K) (Life Technologies) lipoplexes in Opti-MEM (Life Technologies) according to the manufacturer's instructions, at concentrations of 100, 50, and 25 nM for initial screening. PMO solutions were warmed for 5 minutes at 37° C. before being transfected into cells with Endo-Porter® (Genetools) according to the manufacturer's instructions at the concentration of 20 μM. Culture medium was replaced with fresh 10% FBS DMEM before adding the desired volume of PMO and 3 μL of Endo-Porter® for every 500 μL culture medium. Cells were incubated for 24 hours for 2′OMe AO transfections and 72 hours for PMO transfections before RNA was harvested.

### RNA Extraction and PCR

The MagMax™-96 total RNA isolation kit (Life Technologies) was used to extract total RNA from cultured cells, according to the manufacturer's guidelines. RT-PCR was performed using approximately 50 ng of total RNA with Superscript III One-Step RT-PCR System with Platinum® Taq DNA Polymerase (ThermoFisher Scientific, Australia). The PCR conditions used were 55° C. for 30 min, 94° C. for 2 min, followed by 32 cycles of 94° C. for 30 seconds, 60° C. for 1 minute, and 68° C. for 2 min. Primers (PRKN_Ex1F: 5′-TGGAGGATTTAACCCAGGAG-3′; PRKN_Ex3F: 5′-ATGAATGCAACTGGAGGCGA-3′; PRKN_6R: 5′-GACGTCTGTGCACGTAATGC-3′) were designed by NCBI primer blast and supplied by Integrated DNA Technologies, USA. RT-PCR products were resolved on a 2% agarose gel in TAE buffer using a 100 bp DNA ladder (ThermoFisher Scientific) as the size standard. The sizes of full length, exon 4, exon 3 and exons 3+4 excised PRKN mRNA transcript products are 833 bp, 711 bp, 592 bp and 470 bp, respectively. RT-PCR products of interest were band-stabbed, or excised for purification by either Diffinity Rapid Tip™ (Diffinity Genomics, Pennsylvania, USA) or NucleoSpin® Gel and PCR Clean-up (Scientifix Life, Melbourne, Australia), respectively, according to the manufacturer's instructions. Purified PCR products were sent to the Australian Genome Research Facility Ltd. (Nedlands, Australia) for Sanger sequencing.

cDNA Synthesis and Real-Time PCR

The SuperScript™ IV First-Strand Synthesis System (ThermoFisher Scientific) was used to synthesize cDNA. 5 μl of the total RNA was mixed with 0.5 μl random hexamers (100 ng/μl) and 1 μl dNTPs (5 mM) and heated to 65° C. for 5 mins, followed by 5 mins on ice. 2 μl 5× first strand buffer, 0.5 μl 0.1M DTT, 0.5 μl RNase 1 out and 0.5 μl Superscript IV reverse transcriptase (200 units/μl) was added into the mixture before the cDNA synthesis following these conditions: 23° C. for 10 mins, 50° C. for 10mins and 80° C. for 10mins. Real-time PCR (qPCR) reactions were performed by C1000™ thermal cycler (Bio-Rad) using Fast SYBR™ Green Master Mix (ThermoFisher Scientific). Each PCR reaction system (10 μl) contained 5 μl 2× SYBR Green Master Mix, 400 nM p53 primers (p53_Ex1/2qF: 5′-GCTTCCCTGGATTGGCAGC-3′; p53_Ex2qR: 5′-GACGCTAGGATCTGACTGCG-3′) or 500 nM TBP primers (TBP_Ex1/2qF: 5′-TCTTTGCAGTGACCCAGCATCAC-3′; TBP_Ex2qF: 5′-CCTAGAGCATCTCCAGCACACTCT-3′), 3 μl cDNA and H2O (if needed to make a final reaction volume of 10 μl). The qPCR reactions were conducted in triplicate. The cycling conditions consisted of one single step of 95° C. for 1 min followed by 40 cycles (95° C. for 20 seconds, 60° C. for 20 seconds and 72° C. for 20 seconds). A final melting program ranging from 65° C. to 95° C. with a heating rate of 0.5° C. per 10 s was performed to create the melt curve. Negative controls with no cDNA template were included. Standard curves for both p53 and TBP were made to test the efficiency of the primers as well as for the qPCR analysis. PCR efficiency and Ct value were analysed by using Bio-Rad CFX manager 2.1 according the the guidelines provided.

### Western Blot

Western blotting was performed using a protocol derived from Cooper et. al 2003 and Nicholson et. al 1989, 1992. Cells were harvested and resuspended in treatment buffer (100 μI/4.5 mg wet pellet weight) consisting of 125 mmol/L Tris-HCl pH 6.8, 15% sodium dodecyl sulfate, 10% glycerol, 0.5 mmol/L phenylmethylsulfonyl fluoride, 50 mmol/L dithiothreitol, bromophenol blue (0.004% w/v), and a protease inhibitor cocktail (3 μl/100 μl of treatment buffer) (Sigma-Aldrich®, Castle Hill, Australia). Samples were vortexed briefly, sonicated for 1 second, 4-8 times at a setting of 30/100 on an ultrasonic processor (Sonics, Newtown, Conn.) and heated at 95° C. for 5 minutes. Total protein concentration was determined by Pierce BCA protein assay kit (Thermo Fisher Scientific) without bromophenol blue and dithiothreitol. Approximately 15 μg of total protein was loaded for each sample on the NuPage 4-12% Bis/Tris gradient gel (Life Technologies) with 7 μl of Kaleidoscope™ (Bio-Rad) and 3 μI of MagicMark (Thermo Fisher Scientific). Electrophoresis was carried out at 200 V for 1 hour in 1× NuPage MOPS SDS running buffer. The fractionated protein was transferred to a methanol pre-treated polyvinylidene difluoride membrane at 350 mA for 1 hour in western transfer buffer. The membrane was blocked by 5% skim milk in TBS-T for 1 hour before incubated with MAB5512 (1:500, Merck, Castle Hill, Australia) and anti-β-tubulin antibody (1:1000, Developmental Studies Hybridoma Bank, USA) at room temperature for 1 hour. Horseradish peroxidase goat anti-mouse (1:10,000) and goat anti-rabbit (1:10,000) secondary antibodies were incubated for 1 hour before western blot images were detected Fusion FX system (Vilber Lourmat, Marne-la-Vallée, France) using Fusion-Capt software.

### Immunofluorescence

Three days after the 20 μM PMO transfection, patient fibroblasts grown on glass cover slips were treated with 50 μM carbonyl cyanide m-chlorophenyl hydrazone (CCCP) for two hours to depolarize the mitochondria. The glass cover slips were collected and fixed in acetone/methanol (1:1) on ice and 20% goat serum in 0.2% Triton-X in 1× PBS was used to block the slides for 1 hour. Parkin was probed with MAB5512 (1: 200, Merck) for one hour and Alexa Fluor mouse 488 (1:400, Thermo Fisher Scientific) for a further hour. Tomm20 was probed with HPA011562 (1: 100, Sigma-Aldrich) at room temperature for 1 hour and Alexa Fluor rabbit 568 (1:400, Thermo Fisher Scientific) for another hour. Nuclei were stained with Hoechst (1:160, Sigma-Aldrich). Images were captured and analysed using the Nikon Eclipse 80i microscope and the NIS elements program.

### Results

### Antisense Oligomers Designed to Anneal to Acceptor/Donor Splice Sites or Exon Splicing Enhancers

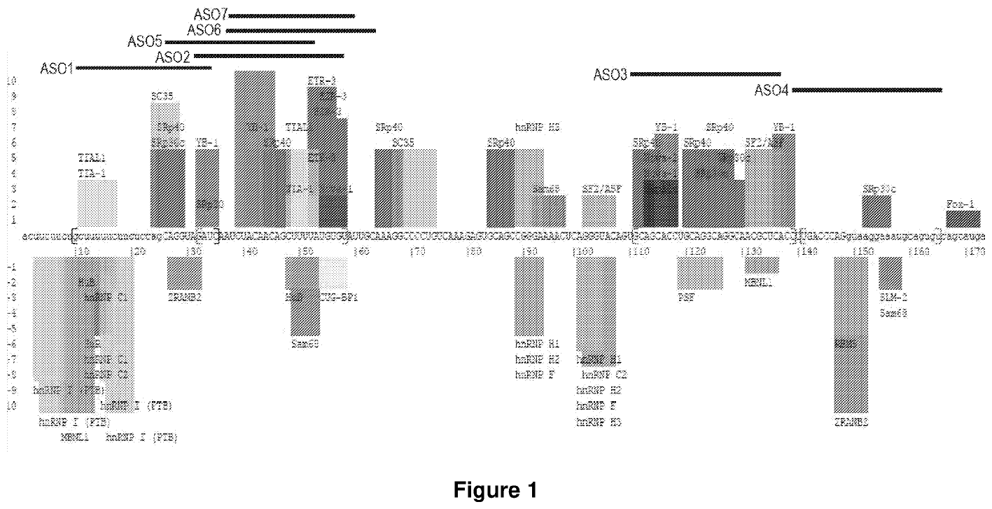

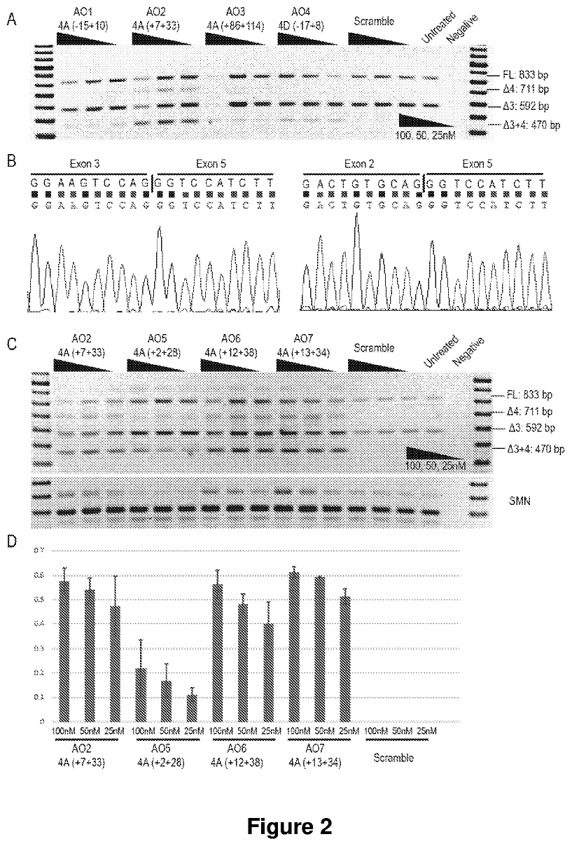

Antisense compounds mediate exon skipping through steric blocking of motifs implicated in exon recognition and processing, such as the acceptor/donor splice sites, or exon splicing enhancers. To induce skipping PRKN of exon 4, we designed a panel of AOs targeting the acceptor or donor splice sites, or exon splicing enhancers as, predicted by SpliceAid (http://www.introni.it/splicing.html) (FIG. 1). Four AOs (Table 1) were designed and synthesised as 2′OMe AO for initial evaluation in patient or normal fibroblasts, where RT-PCR across exons 1-6 was carried out on RNA extracted from transfected cells to assess levels of exon 4 skipping (FIG. 2A). After identifying the most promising sequence to induce exon 4 skipping as, AO2_H4A (+7+33), further refinement was undertaken by “micro-walking” to generate additional overlapping AOs 5-7 targeting this region (FIG. 1).

### Antisense Oligonucleotide-Induced Exon 4 Skipping Restores the PRKN Reading Frame in Patient Fibroblasts

The PRKN mutations in a PD patient were confirmed by Sanger sequencing, Deletion of exon 3 was identified on one allele and a single base substitution (c.719 C>T) in exon 6 on the second allele (Supplementary FIG. 1). The first-generation AOs targeting PRKN exon 4 induced variable levels of exon 4 skipping, with three AOs inducing detectable exon 4 skipping, and AO2_H4A(+7+33) demonstrating the highest exon skipping efficiency of approximately 30% when transfected at 25 nM (FIG. 2A). As the full-length product and the transcript product with the exon 3 genomic deletion are of similar abundance, nonsense mediated decay does not appear significant in this case. All shortened products were confirmed by Sanger sequencing (FIG. 2B) and found to be missing exon 4 from the allele carrying the c.719 C>T missense mutation in exon 3, and exons 3 and 4 from the allele carrying the exon 3 genomic deletion. In the initial screen, AO2_H4A(+7+33) showed the highest exon skipping efficiency of approximately 30% when transfected at 25 nM (FIG. 2A). Subsequent design of overlapping AOs refined the most efficient AO sequence as AO7_H4A(+13+34) that induced robust exon 4 skipping in a dose-response manner (FIG. 2C), with approximately 50% exon skipping at 25 nM, as assessed by densitometry (FIG. 2D). As expected, the scrambled control sequence had no effect on PRKN exon 4 selection in normal (not shown) or PRKN patient cells.

### Morpholino Oligomer-Induced PRKN Exon 4 Skipping and Production of a Shorter Functional Parkin Isoform

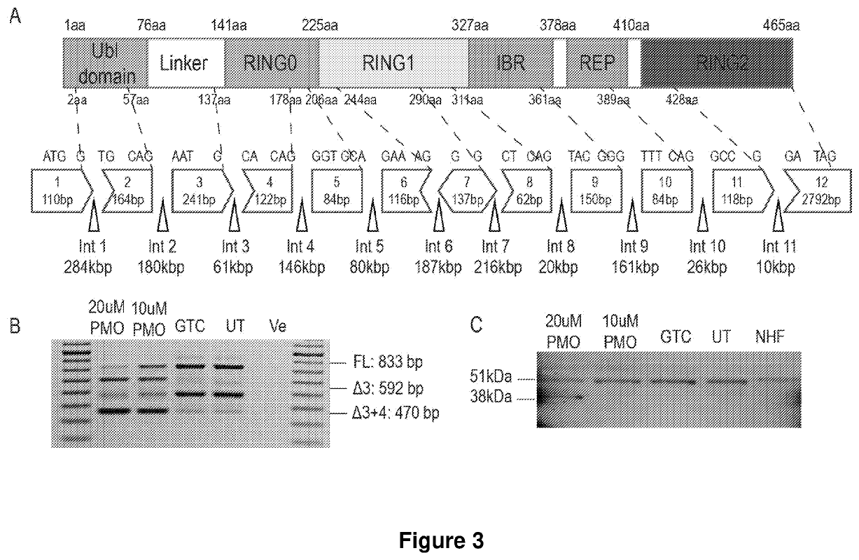

As shown in FIG. 3A, generation of 711 and 470 bp amplicons by RT-PCR indicates that robust exon 4 skipping from both PRKN transcripts was induced by the PMO H4A(+13+34). The PMO-induced exon 4 skipping is clearly in excess of the low levels of endogenous PRKN exon 4 skipping seen in untreated or sham transfected patient cells. The removal of PRKN exon 4 from the mRNA in patient fibroblasts with genomic deletion of exon 3 restores the PRKN reading frame, allowing translation of an internally deleted, shorter parkin protein (FIG. 3B). The shorter in-frame transcript missing exons 3 and 4 can now be translated into an internally truncated 38 kDa protein missing 121 amino acids (aa) (58-178 aa) as shown in FIG. 3C. No truncated protein was observed in the scramble sequence transfected or untreated patient fibroblasts.

### PMO Induced Shorter Parkin Protein Retains Some Function

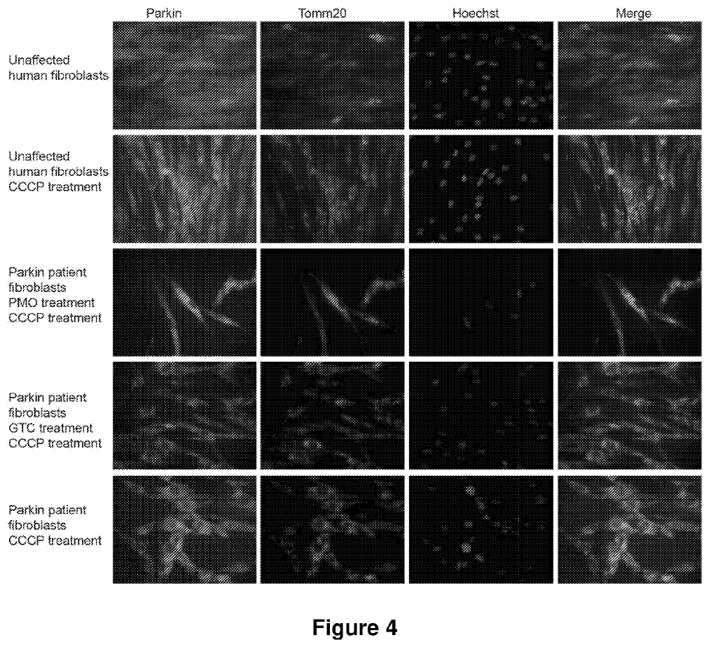

Patient fibroblasts were treated with 20 μM PMO H4A(+13+34) and incubated for 72 hours, before depolarization of the mitochondria by 50 μM carbonyl cyanide m-chlorophenyl hydrazone (CCCP) treatment for two hours. As shown in FIG. 4, parkin protein is typically located uniformly in the cytoplasm in normal human fibroblasts without any treatment. However, after 50 μM CCCP treatment, cytoplasmic healthy-type parkin protein colocalises with depolarised mitochondria in the perinuclear region. A similar pattern of parkin protein location was shown in PD patient fibroblasts treated with PMO, but was not evident in gene-tools control PMO treated or untreated PD patient fibroblasts.

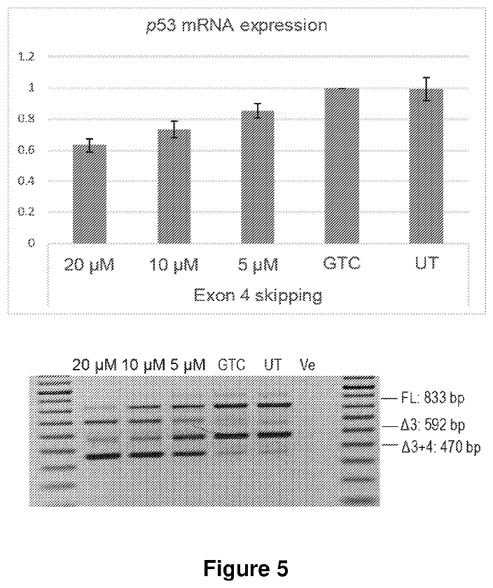

RNA was extracted from the parkin patient cultured cells and cDNA was synthesized for the real-time qPCR analysis in order to compare p53 expression levels before and after the PMO treatment. As shown in FIG. 5, there was approximately 40% reduction of p53 expression in cells treated with 20 μM PMO H4A(+13+34) for 72 hours, compared to those treated with the Gene-tools control PMO and to untreated patient cells. A dose response of p53 expression to PMO H4A(+13+34) was also observed, which corresponded to the exon skipping levels as determined by RT-PCR.

### 2′OMe AOs Cocktails Mediated PRKN Exon 3 Skipping in Healthy Human Fibroblasts

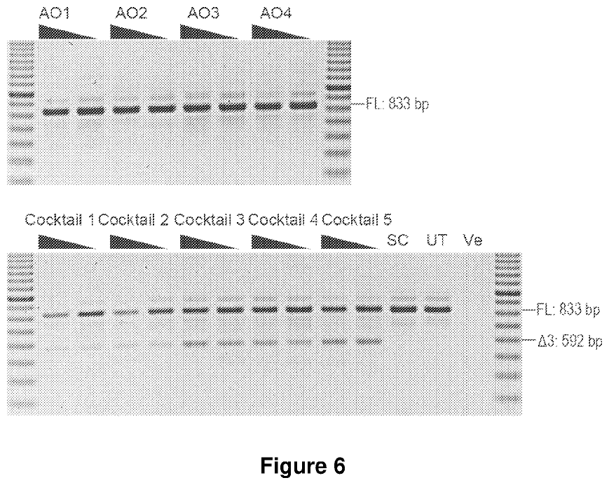

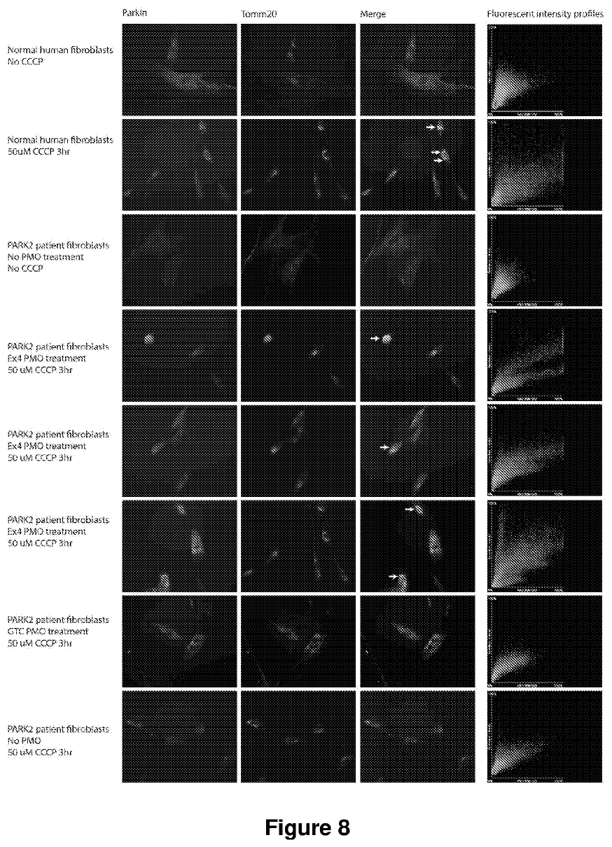

We designed and synthesised PRKN exon 3 skipping AOs to be used in conjunction with PMO H4A(+13+34) as a potential therapy for patients carrying an exon 4 genomic deletion or those with pathogenic intra-exonic mutations in exons 3 or 4. Fourteen AO sequences covering exon 3 and the exon-intron boundaries (FIG. 8) were transfected in healthy human fibroblasts. None of the individual AO transfections induced exon 3 skipping when analysed by RT-PCR (data not shown), while some combinations of two AOs transfected into the healthy human fibroblasts as “cocktails” did induce minimal exon 3 skipping (data not shown). Three AO combinations were then transfected into the healthy human fibroblasts. Cocktail 47 and cocktail 48 induced approximately 30% skipping of exon 3, while slightly increased exon 3 skipping (35%) was demonstrated when four AOs were combined as cocktail 51 (FIG. 6).

TABLE 2
Antisense oligonucleotide sequence efficacy
score for AO-induced parkin exon skipping.

| | | Length | Efficacy |
|---|---|---|---|
| Seq ID | AO nomenclature | (bp) | score |
| 1 | PRKN_H2A (−16+7) | 23 | 0 |
| 2 | PRKN_H2A (+51+78) | 27 | 0 |
| 3 | PRKN_H2A (+116+141) | 26 | 1 |
| 4 | PRKN_H2D (+22−4) | 26 | 0 |
| 5 | PRKN_H2A (+10+33) | 24 | 0 |
| 6 | PRKN_H3A (+9+35) | 27 | −1 |
| 7 | PRKN_H3A (+205+230) | 25 | −1 |
| 8 | PRKN_H3A (−5+22) | 27 | −1 |
| 9 | PRKN_H3D (−5+21) | 25 | −1 |
| 10 | PRKN_H3A (+137+161) | 25 | −1 |
| 11 | PRKN_H4A (−15+10) | 25 | 0 |
| 12 | PRKN_H4A (+7+33) | 27 | 0 |
| 13 | PRKN_H4A (+86+114) | 29 | 0 |
| 14 | PRKN_H4D (−17+8) | 25 | 0 |
| 15 | PRKN_H2A (+111+136) | 26 | 1 |
| 16 | PRKN_H2A (+121+146) | 26 | 0 |
| 17 | PRKN_H2A (+118+139) | 22 | 0 |
| 18 | PRKN_H3A (+36+60) | 25 | −1 |
| 19 | PRKN_H3A (+62+86) | 25 | −1 |
| 20 | PRKN_H3A (+50+69) | 20 | −1 |
| 21 | PRKN_H3A (+55+81) | 26 | −1 |
| 22 | PRKN_H3A (+195+219) | 25 | −1 |
| 23 | PRKN_H3D (−20+5) | 25 | −1 |
| 24 | PRKN_H4A (+2+28) | 27 | 1 |
| 25 | PRKN_H4A (+12+38) | 27 | 1 |
| 26 | PRKN_H4A (+13+34) | 22 | 1 |
| 27 | PRKN_H3A (+26+34, +63+71, +209+218) | 27 | −1 |
| 28 | PRKN_H3A (+20+32, +63+75) | 25 | −1 |
| 29 | PRKN_H3A (+63+75, +201+213) | 25 | −1 |
| 30 | PRKN_H3A (+20+31, +201+213) | 25 | −1 |
| 31 | PRKN_H4A (+13+34) | 22 | 1 |

+1: >50% exon skipping,
0: <50% exon skipping,
−1: No exon skipping

### REFERENCES

Mann, C. J., Honeyman, K., McClorey, G., Fletcher, S. & Wilton, S. D. Improved antisense oligonucleotide induced exon skipping in the mdx mouse model of muscular dystrophy. The journal of gene medicine 4, 644-654, doi:10.1002/jgm.295 (2002).
Ye, J. et al. Primer-BLAST: a tool to design target-specific primers for polymerase chain reaction. BMC bioinformatics 13, 134 (2012).
Wilton, S. D., Lim, L., Dye, D. & Laing, N. Bandstab: a PCR-based alternative to cloning PCR products. Biotechniques 22, 642-645 (1997).
Cooper, S. T., Lo, H. P. & North, K. N. Single section Western blot: improving the molecular diagnosis of the muscular dystrophies. Neurology 61, 93-97 (2003).
Nicholson, L. V. et al. Dystrophin in skeletal muscle. I. Western blot analysis using a monoclonal antibody. J Neurol Sci 94, 125-136 (1989).
Nicholson, L. V. et al. Dystrophin or a “related protein” in Duchenne muscular dystrophy? Acta Neurol Scand 86, 8-14 (1992)

1. An isolated or purified antisense oligomer with a modified backbone structure for modifying pre-mRNA splicing in the parkin gene transcript or part thereof.

2. The antisense oligomer of claim 1 that induces skipping of target exons in the parkin gene transcript or part thereof.

3. The antisense oligomer of claim 1 selected from the list comprising: SEQ ID NOs: 1-31.

4. The antisense oligomer of claim 1 wherein the antisense oligomer contains one or more nucleotide positions subject to an alternative chemistry or modification chosen from the list comprising: (i) modified sugar moieties; (ii) resistance to RNase H; (iii) oligomeric mimetic chemistry.

5. The antisense oligomer of claim 1 wherein the antisense oligomer is further modified by:
(i) chemical conjugation to a moiety; and/or (ii) tagging with a cell penetrating peptide.

6. The antisense oligomer of claim 1 wherein the antisense oligomer is a phosphorodiamidate morpholino oligomer.

7. The antisense oligomer of claim 1 wherein when any uracil (U) is present in the nucleotide sequence, the uracil (U) is replaced by a thymine (T).

8. The antisense oligomer of claim 1 that operates to induce skipping of one or more of the exons of the parkin gene transcript or part thereof.

9. A method for manipulating splicing in a parkin gene transcript, the method including the step of:
a) providing an antisense oligomer according to any one of claims 1 to 8 and combinations or cocktails thereof and allowing the oligomer(s) to bind to a target nucleic acid site.

10. A pharmaceutical, prophylactic, or therapeutic composition to treat, prevent or ameliorate the effects of a disease related to parkin expression in a patient, the composition comprising:
a) an antisense oligomer according to any one of claims 1 to 8 and combinations or cocktails thereof, and
b) one or more pharmaceutically acceptable carriers and/or diluents.

11. A method to treat, prevent or ameliorate the effects of a disease associated with parkin expression, comprising the step of:
a) administering to the patient an effective amount of an antisense oligomer, or pharmaceutical composition comprising an antisense oligomer, according to any one of claims 1 to 9 and combinations or cocktails thereof.

12. The use of an antisense oligomer according to any one of claims 1 to 9 and combinations or cocktails thereof, for the manufacture of a medicament to treat, prevent or ameliorate the effects of a disease associated with parkin expression.

13. A kit to treat, prevent or ameliorate the effects of a disease associated with parkin expression in a patient, which kit comprises at least an antisense oligomer according to any one of claims 1 to 9 and combinations or cocktails thereof, packaged in a suitable container, together with instructions for its use.

14. The composition of claim 11, method of claim 10 or 12, use of claim 13 or kit of claim 14 wherein the parkin expression related disease is Parkin-type autosomal recessive juvenile Parkinson's disease.

15. The composition of claim 11, method of claim 10 or 12, use of claim 13 or kit of claim 14 wherein the subject with the disease associated with parkin expression is a human.

16. The antisense oligomer of claim 1 selected from the list comprising: SEQ ID NOs: 11-14, 24-26 and 31.

17. The antisense oligomer of claim 1 selected from the list comprising: SEQ ID NOs: 24-26 and 31.


SEQUENCE LISTING (38 sequences)

SEQ ID NO: 1 - DNA, 23 bp, Homo sapiens
acaaacacugaccaaggaaauug

SEQ ID NO: 2 - DNA, 27 bp, Homo sapiens
cuccuugagcuggaagaugcugguguc

SEQ ID NO: 3 - DNA, 26 bp, Homo sapiens
cuccuucccugcgaaaaucacacgca

SEQ ID NO: 4 - DNA, 26 bp, Homo sapiens
ucaccugcacaguccagucauuccuc

SEQ ID NO: 5 - DNA, 24 bp, Homo sapiens
ggaaaccauggcuggaguugaacc

SEQ ID NO: 6 - DNA, 27 bp, Homo sapiens
augugaacaaugcucugcugauccagg

SEQ ID NO: 7 - DNA, 26 bp, Homo sapiens
cugguggugaguccuuccugcuguca

SEQ ID NO: 8 - DNA, 27 bp, Homo sapiens
ucugcugauccaggucacaauucuguu

SEQ ID NO: 9 - DNA, 26 bp, Homo sapiens
auuaccuggacuuccagcugguggug

SEQ ID NO: 10 - DNA, 25 bp, Homo sapiens
cugagcugcugagguccacccgagu

SEQ ID NO: 11 - DNA, 25 bp, Homo sapiens
gaucuaccugcuggagaagaaaaag

SEQ ID NO: 12 - DNA, 27 bp, Homo sapiens
acacauaaaagcuguuguagauugauc

SEQ ID NO: 13 - DNA, 29 bp, Homo sapiens
ggugagcguugccugccugcaggugcugc

SEQ ID NO: 14 - DNA, 25 bp, Homo sapiens
acacugcauuuccuuaccuggguca

SEQ ID NO: 15 - DNA, 26 bp, Homo sapiens
ucccugcgaaaaucacacgcaacugg

SEQ ID NO: 16 - DNA, 26 bp, Homo sapiens
cucagcuccuucccugcgaaaaucac

SEQ ID NO: 17 - DNA, 22 bp, Homo sapiens
ccuucccugcgaaaaucacacg

SEQ ID NO: 18 - DNA, 25 bp, Homo sapiens
accuuuucuccacggucucugcaca

SEQ ID NO: 19 - DNA, 25 bp, Homo sapiens
ucgccuccaguugcauucauuucuu

SEQ ID NO: 20 - DNA, 20 bp, Homo sapiens
cauuucuugaccuuuucucc

SEQ ID NO: 21 - DNA, 26 bp, Homo sapiens
ccaguugcauucauuucuugaccuuu

SEQ ID NO: 22 - DNA, 25 bp, Homo sapiens
guccuuccugcugucagugugcaga

SEQ ID NO: 23 - DNA, 25 bp, Homo sapiens
ucuuagagcauuccaauuaccugga

SEQ ID NO: 24 - DNA, 27 bp, Homo sapiens
uaaaagcuguuguagauugaucuaccu

SEQ ID NO: 25 - DNA, 27 bp, Homo sapiens
gcaauacacauaaaagcuguuguagau

SEQ ID NO: 26 - DNA, 22 bp, Homo sapiens
uacacauaaaagcuguuguaga

SEQ ID NO: 27 - DNA, 27 bp, Homo sapiens
cugauccagcgccuccagagugugcag

SEQ ID NO: 28 - DNA, 25 bp, Homo sapiens
gcucugcugaucccgccuccaguug

SEQ ID NO: 29 - DNA, 25 bp, Homo sapiens
cgccuccaguugccugcugucagug

SEQ ID NO: 30 - DNA, 25 bp, Homo sapiens
gcucugcugaucccugcugucagug

SEQ ID NO: 31 - DNA, 22 bp, Homo sapiens
tacacataaaagctgttgtaga

SEQ ID NO: 32 - DNA, 20 bp, Homo sapiens
tggaggatttaacccaggag

SEQ ID NO: 33 - DNA, 20 bp, Homo sapiens
atgaatgcaactggaggcga

SEQ ID NO: 34 - DNA, 20 bp, Homo sapiens
gacgtctgtgcacgtaatgc

SEQ ID NO: 35 - DNA, 19 bp, Homo sapiens
gcttccctggattggcagc

SEQ ID NO: 36 - DNA, 20 bp, Homo sapiens
gacgctaggatctgactgcg

SEQ ID NO: 37 - DNA, 23 bp, Homo sapiens
tctttgcagtgacccagcatcac

SEQ ID NO: 38 - DNA, 24 bp, Homo sapiens
cctagagcatctccagcacactct